(19) United States
(12) Patent Application Publication
(10) Pub. No.: US 2009/0143823 A1
(43) Pub. Date: Jun. 4, 2009

(54) Transverse connector system for spinal rods

(76) Inventors: Dong M. Jeon, Draper, UT (US); Patrick D. Moore, West Jordan, UT (US); Sang K. Lee, Seoul (KR)

Correspondence Address: MORRISS OBRYANT COMPAGNI, P.C., 734 EAST 200 SOUTH, SALT LAKE CITY, UT, 84102, US

(21) Appl. No.: 12/313,019

(22) Filed: Nov. 13, 2008

Related U.S. Application Data
(60) Provisional application No. 60/987,962, filed on Nov. 14, 2007.

(51) Int. Cl. A61B 17/70

(52) U.S. Cl. 606/250; 606/278

(57) ABSTRACT

A transverse connector system for coupling elongate elements to each other in a spinal fixation system. An upper arm member and a lower arm member are connected by a washer and locking bolt to form a transverse plate. The distal ends of the upper arm member and the lower arm member each include a swivel rod connector and may be attached to a locking rod connector using a locking bolt to secure an elongate fixation element therein. The upper arm member and lower arm member are laterally adjustable and pivotably adjustable with respect to one another due to the washer and slot connection, and thus accommodate divergences and convergences in the attached rods.

### CROSS-REFERENCE TO RELATED APPLICATION

This application claims the benefit of U.S. Provisional Application No. 60/987,962, filed Nov. 13, 2007, the disclosure of which is incorporated herein by reference in its entirety.

### TECHNICAL FIELD

The present invention relates to a device for spinal fixation, and in particular to a transverse connector system for coupling spinal rods, plates, or other elongate members.

### BACKGROUND

It is often necessary to surgically treat spinal disorders such as scoliosis. Numerous systems for use in spinal correction and fixation have been disclosed. These systems usually include a pair of elongate members, typically either rods or plates, placed along the vertebral column. For the sake of simplicity, the term “rod” is used throughout to refer to any such elongate member. Each rod is attached to the spine with various attachment devices. These attachment devices may include, but are not limited to, pedicle screws, plates, transverse process hooks, sublaminar hooks, pedicle hooks, and other similar devices.

It is also well known that the strength and stability of a dual rod assembly can be increased by coupling two rods with a cross-brace or transverse connector which extends substantially horizontal to the longitudinal axes of the rods, typically across the spine. The simplest situation in which a transverse connector may be used occurs when the two rods are geometrically aligned parallel to each other in all three dimensions. In such an alignment, there is no convergence or divergence between the rods in the medial-lateral direction, the two rods have the same orientation with respect to the coronal plane (viewed in the anterior-posterior direction), the rods are coplanar from a lateral view, and the two rods are located a uniform distance from each other.

However, the two rods are rarely three dimensionally geometrically aligned in clinical situations. There are several ways to address the variations of geometrical alignment. First, one or both of the rods can be bent to accommodate a transverse connector. However, any bending in either of the rods can adversely affect the fixation to the spine and compromise the clinical outcome. Furthermore, such bending can also adversely affect the mechanical properties of the rods. Alternatively, a transverse connector can be bent so that disturbance to the rod positioning is minimized. As is the case with bending of the rods, the mechanical properties of the transverse connector may be compromised by such bending.

In order to address this issue, transverse connectors with some adjustability have been designed to adapt for variations from geometrical alignment. However, most of such connectors are multi-piece systems which can be difficult to assemble and use in the surgical environment. For example, U.S. Pat. No. 5,980,523, the disclosure of which is incorporated herein by reference in its entirety, discloses a multi-piece transverse connector for spinal rods that can accommodate converging or diverging rods. However, accidental disassembly of this type of connector by the surgeon is possible.

Other connectors which are one-piece designs do not allow for adjustments to compensate for all three modes in which there may be variation from geometrical alignment: convergence or divergence, non-coplanar rods, and variability in rod separation distances. For example, U.S. Pat. No. 5,947,966, the disclosure of which is incorporated by reference herein, discloses a device for linking adjacent spinal rods. In one embodiment, the device includes two members that are movable with respect to one another to accommodate different rod separation distances. A pin on one member engages a groove on the other member to provisionally couple the two members, thereby preventing a surgeon from separating the two members. Because the pin is sized to exactly fit the groove, no movement of the pin transverse to the longitudinal axis of the groove is possible. As a result, the devices similar to the '966 patent device cannot accommodate non-coplanar rods or adjust for rod convergence or divergence.

Thus, there exists a need for an improved transverse connector utilized for connecting or coupling elongate fixation elements to each other in a spinal fixation system that allows for adjustment in translational and/or rotational placement to adjust for convergence or divergence, non-coplanarity, and variability in separation between the elongate fixation elements.

### SUMMARY

In one illustrative embodiment, the present invention includes a transverse connector system for coupling first and second elongate spinal fixation elements which may have different three dimensional orientations to each other. An upper arm member and a lower arm member are connected by a washer and locking bolt to form a transverse plate. The distal ends of the upper arm member and the lower arm member each include a swivel rod connector and may be attached to a locking rod connector using a locking bolt to secure an elongate fixation element therein. The upper arm member and lower arm member are laterally adjustable and pivotably adjustable with respect to one another due to the washer and slot connection, and thus accommodate different separation distances and orientations between elongate fixation elements in a spinal fixation system.

Additional embodiments, examples, advantages, and objects of the present invention will be apparent to those of ordinary skill in the art from the following specification.

### DESCRIPTION OF THE DRAWINGS

It will be appreciated by those of ordinary skill in the art that the elements depicted in the various drawings are not to scale, but are for illustrative purposes only. The nature of the present invention, as well as other embodiments of the present invention may be more clearly understood by reference to the following detailed description of the invention, to the appended claims, and to the several drawings attached hereto.

FIGS. 1A to 1C are top, partial cut-away side, and bottom views of one illustrative embodiment of a transverse-connector system in accordance with the principles of the present invention.

FIGS. 2A through 2E are top, front, bottom, right side, and left side views of an upper arm member of the transverse-connector system of FIG. 1.

FIGS. 3A through 3E are top, front, bottom, right side, and left side views of a lower arm member of the transverse-connector system of FIG. 1.

FIGS. 4A through 4C are top, side cut-away, and bottom views of a lock washer of the transverse-connector system of FIG. 1.

FIGS. 5A through 5C are bottom, side, and top views of a locking bolt of the transverse-connector system of FIG. 1.

FIGS. 6A through 6C are top, sectional side, and bottom views of a locking rod connector arm of the transverse-connector system of FIG. 1.

FIGS. 7A through 7C are top, side, and bottom views of a swivel rod connector of the transverse-connector system of FIG. 1.

FIGS. 8A and 8B are top and side views of a pin of the transverse-connector system of FIG. 1.

### DETAILED DESCRIPTION

For the purposes of promoting an understanding of the principles of the invention, reference will now be made to the embodiment illustrated in the drawings and specific language will be used to describe the same. It will nevertheless be understood that no limitation of the scope of the invention is thereby intended, such alterations and further modifications in the illustrated device, and such further applications of the principles of the invention as illustrated therein being contemplated as would normally occur to one skilled in the art to which the invention relates.

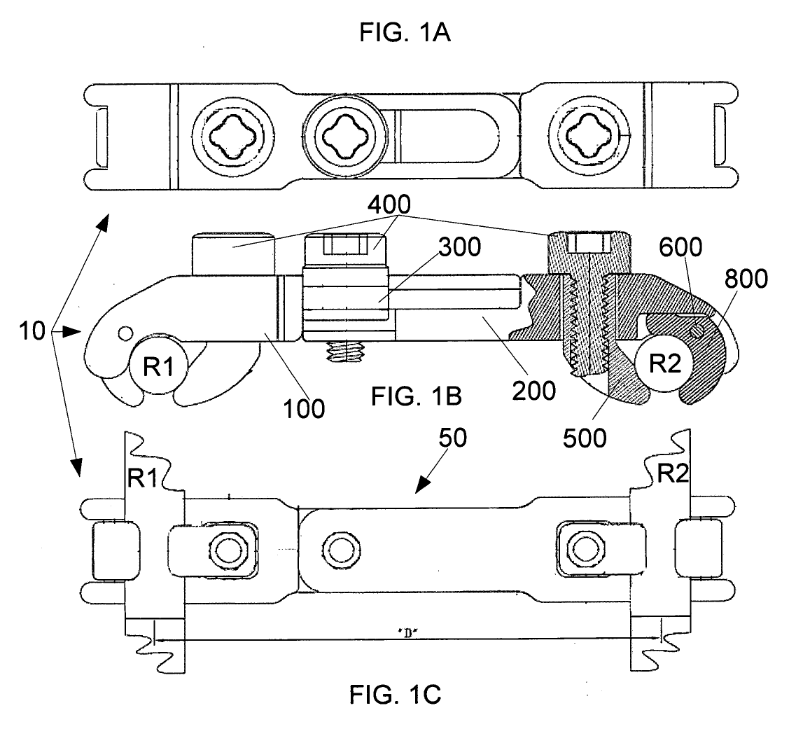

Referring generally to FIG. 1, a transverse connector system 10 in accordance with the present invention is depicted. Transverse-connector system 10 may include an upper arm member 100 and a lower arm member 200. The medial portions of the upper arm member 100 and lower arm member 200 are connected by a locking washer 700 and locking bolt 400 to form a transverse plate 50. The distal ends of each of upper arm member 100 and a lower arm member 200 include a swivel rod connector 800 and may be attached to a locking rod connector 600 using a locking bolt 500 to secure an elongate fixation element therein.

As shown, transverse connector system 10 may be used for coupling a first elongate fixation element R1 to a second elongate fixation element R2. First and second elongate fixation elements R1 and R2 may be cylindrical rods, rectangular bars, plates, or any other device suitable for spinal fusion. In a spinal fixation application, first elongate fixation element R1 may extend along one side of the vertebral column, attached thereto by separate attachment elements. Similarly, second elongate fixation element R2 may extend along the opposite side of the vertebral column, attached thereto by separate attachment elements.

The components of transverse-connector system 10 can be made of any sturdy biocompatible material suitable for an orthopedic application. Suitable materials may include titanium, stainless steel, and alloys containing the same. Where the components of transverse-connector system 10 are constructed from metallic materials, the materials may be similar, or identical to, the metallic materials used for the elongate fixation elements to avoid galvanic (mixed-metal) corrosion.

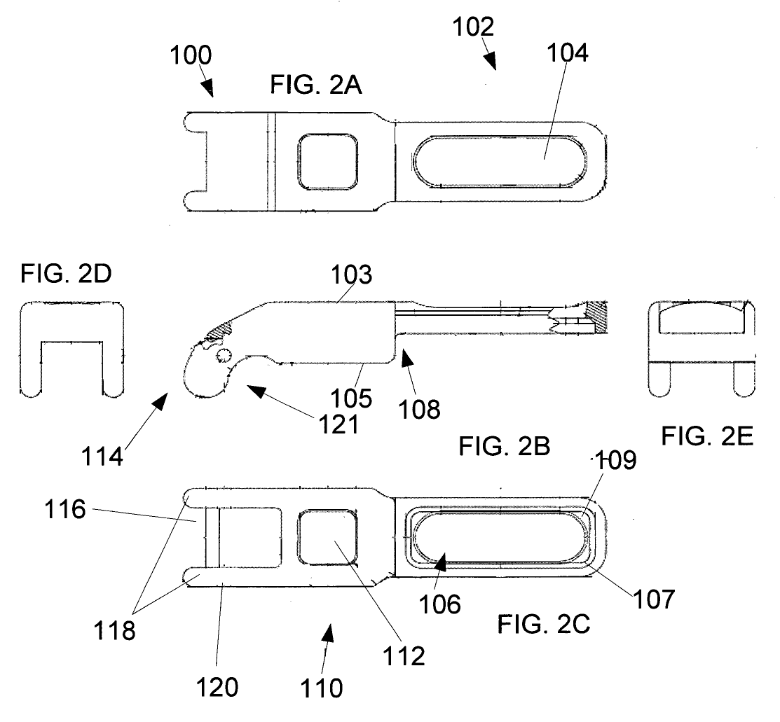

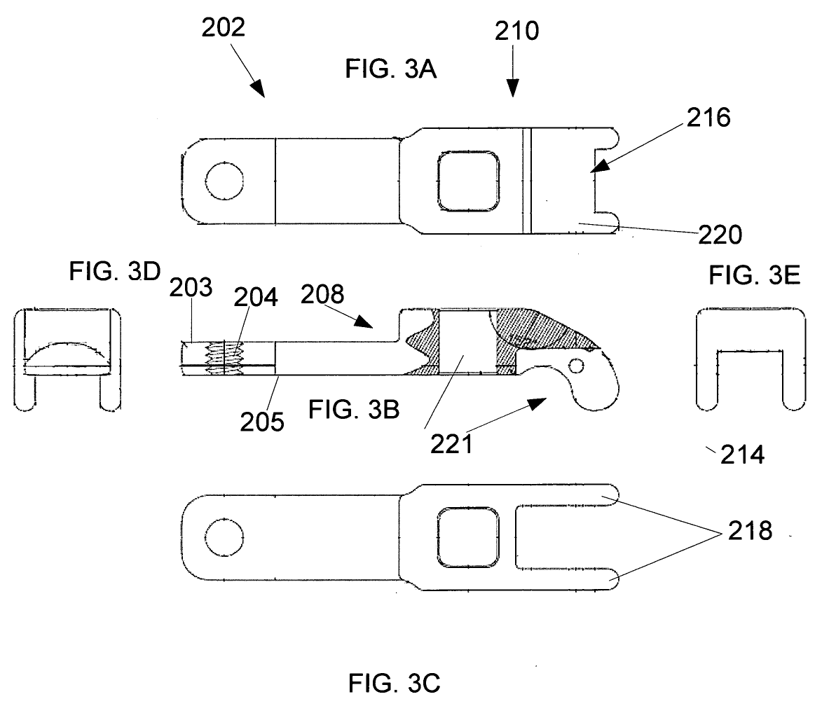

As shown in FIGS. 1A through 2C, upper arm member 100 has a plate portion 102, which in connection with the plate portion 202 of the lower arm member (see, FIGS. 3A to 3C) forms the transverse plate 50, and a rod connection portion 110.

Plate portion 102 is thinner than rod connection portion 110, which is formed by having an undercut 108 or step forming a recess in the lower surface 105. An adjustment slot 104 is formed as an elongated slot that passes from upper surface 103 to lower surface 105 of plate portion 102. An inset recess 106 may be formed in lower surface 105 surrounding the adjustment slot 104. In some embodiments, inset recess 106 may have a substantially vertical wall 107 topped by a slanted or concave portion 109. The surface of inset 106 may be roughened or knurled to increase engagement to other components of the system 10, as discussed further herein. In some embodiments, the entirety of the lower surface 105 in plate portion 102 may be roughened or knurled.

Rod connection portion 110 includes a locking connector port 112, which may be formed as an opening passing generally from upper surface 103 to lower surface 105 and having a generally rectangular cross-section with rounded comers. Distal to the locking connector port 112, upper arm 100 terminates in a curved distal end 114, curving downwards from the general plane of upper surface 103 to terminate in two side arms 118 with a recess 116 for a swivel rod connector. Each of arms 118 has a port 120 to allow a pin 600 to be inserted therein for attachment of a swivel rod connector 800. A rod recess 121 maybe formed in lower surface 105 adjacent the arms 118.

As shown in FIGS. 1A through 1C and 3A through 3C, lower arm member 200 has a plate portion 202, which in connection with the plate portion 102 of the upper arm member forms the transverse plate 50, and a rod connection portion 210.

Plate portion 202 is thinner than rod connection portion 210, which is formed by having a cut or step 208 forming a recess in the upper surface 203. A bolt hole 204 for a locking bolt 400 is formed as a threaded opening that passes from upper surface 203 to lower surface 205 of plate portion 202. It will be appreciated that in some embodiments, bolt hole 204 may not be threaded and a threaded nut may be used underneath lower surface 205. In some embodiments, the upper surface 203 in plate portion 202 may be roughened or knurled.

Rod connection portion 210 mirrors rod connector portion 110 of the upper arm member, including a locking connector port 212, which may be formed as an opening passing generally from upper surface 203 to lower surface 205 and having a generally rectangular cross-section with rounded corners. Distal to the locking connector port 212, lower arm 200 terminates in a curved distal end 214, curving downwards from the general plane of upper surface 203 to terminate in two side arms 218 with a recess 216 for a swivel rod connector 800. Each of arms 218 has a port 220 to allow a pin 600 to be inserted therein for attachment of a swivel rod connector 800. A rod recess 221 may be formed in lower surface 205 adjacent the arms 218.

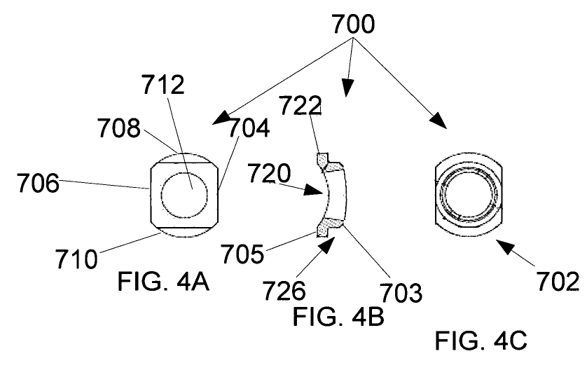

Washer 700 is best depicted in FIGS. 4A through 4C. Washer 700 is formed as a body 702 having a bore 712 passing from an upper surface 703 to a lower surface 705. The walls of bore 712 may have a smooth surface. The outer sidewall of washer 700 has opposite parallel portions 704 and 706, with a vertical sidewall portion 726 sized to fit within the inset recess 106 vertical walls 107 of the upper arm 100, which are joined by two curved portions 708 and 710. The upper surface 703 is convex or angled to match the angled or convex portion 109 of inset recess 106. A lower flange 722 surrounds the lower edge of the washer 700 forming a step to sidewalls 726. Lower surface 705 includes a concave portion 720 inside flange 722.

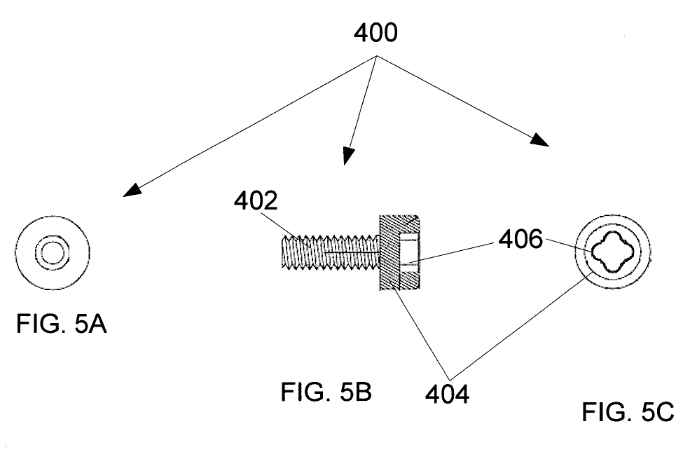

FIGS. 5A through 5C depict a locking bolt 400 for use in the transverse connector system 10. A threaded shaft 402 extends from an enlarged head 404, which may have a generally planar bottom surface. Head 404 may have a generally planar upper surface that features a tool engaging print 406, allowing a tool to be used to rotate the bolt 400 for attachment. It will be appreciated that although three identical bolts are depicted, embodiments using different sized bolts 400 are contemplated and within the scope of the present invention.

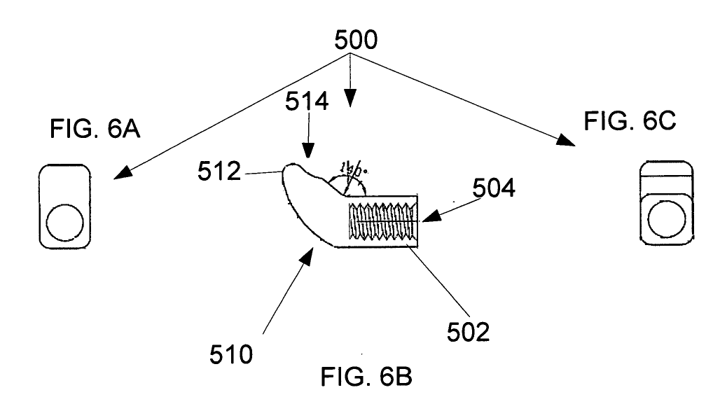

FIGS. 6A through 6C depict a locking rod connector 500 for use in the transverse connector system 10. A locking portion 502 is formed as an elongated body having a generally rectangular cross-section with rounded corners, sized to be received in locking connector port 112 or 212. A threaded hollow shaft 504 opens at the upper surface and passes into the locking portion 502. A rod connection portion 510 angles distally from the locking position to a curved tip 512, forming an angle of about 140 degrees. A slight recess 514 may be formed in the inner face 510 of the connection angled portion.

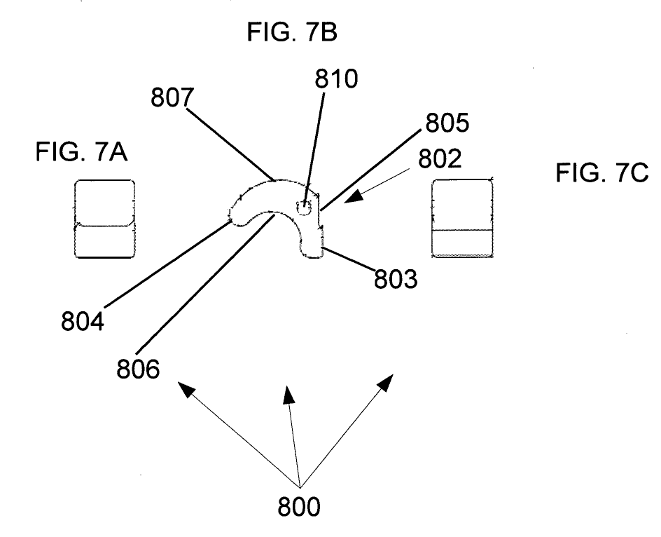

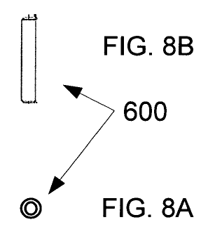

FIGS. 7A through 7C depict one embodiment of a swivel rod connector 800 for use in the transverse connector system 10. Swivel rod connector 800 is formed as a member having a top surface 802 with a proximal first plane 803 stepping down to a distal second plane 805. Outer surface 807 curves convexly from second plane 805 to a tip 804 and inner surface 806 curves the generally opposite concavity to parallel first plane 803 at an upper end of the proximal bottom surface. The inner surface 806 may have a generally flat sidewall throughout the curved surface. A hole 810 passes through the swivel rod connector 800 parallel to the inner surface 806. A pin 600 (FIGS. 8A and 8B) formed as a cylinder may be used to connect the swivel rod connector 800 to a upper arm member 100 or lower arm member 200, passing through hole 810 into the ports 120 or 220 of the arms 118 or 218 to retain the connector 800 within the recess 116 or 216.

To connect a first elongate fixation element R1 to a second elongate fixation element R2, such as cylindrical rods, extending along either side of the vertebral column, a transverse connector system 10 may be installed over the rods R1 and R2 at an angle generally perpendicular to the long axis of the rods. Lower arm member 200 may be connected to a rod R2 by placing over the rod to pivot swivel connector 800 on pin 600 about the axis of the spinal rod. A locking rod connection member 500 is then placed into the locking rod connection port 212 from the bottom and drawn against rod R2 by threading a bolt 400 therethrough from top surface 203 of the lower arm member 200. This process may be repeated with upper arm 100 and the other rod R1. Washer 700 is placed atop lower arm member 200 over bolt hole 204 and upper arm member 100 is placed thereover such that washer 700 resides in the recess 106. The slot 104 allows for expansion and contraction allowing the transverse connector system 10 to adjust to variabilities in the distance D (FIG. 1) between the first elongate fixation element R1 and the second elongate fixation element R2. Washer 700 also allows for pivoting the arm members 100 and 200 with respect to one another due to the cylindrical upper end and convexed lower end allowing them to adjust to variability due to the convergence or divergence of the first elongate fixation element R1 to the second elongate fixation element R2. A third bolt 400 is placed through slot 104 from upper surface 103, passing through washer 700 into bolt hole 204. Once the system is adjusted to a final position, the bolts 400 are tightened, with the middle bolt being tightened last.

While the present invention has been shown and described in terms of preferred embodiments thereof, it will be understood that this invention is not limited to any particular embodiment and that changes and modifications may be made without departing from the true spirit and scope of the invention as defined and desired to be protected.

What is claimed is:

1. A transverse connector system for coupling elongate elements to each other in a spinal fixation system, comprising:
an upper arm member with an upper plate portion and a first connection end that includes a first swivel rod connector and a first locking rod connector;
a lower arm member with an lower plate portion and a second connection end that includes a second swivel rod connector and a second locking rod connector, the upper arm member connected to the lower arm member, such that the first connection end and second connection end are laterally adjustable and pivotably adjustable with respect to one another to accommodate divergences and convergences in two parallel attached elongate elements that may be retained in the first connection end and second connection end.

2. The system of claim 1, further comprising a washer with at least one convex exterior surface disposed between the upper arm member and the lower arm member and a locking bolt, wherein the locking bolt passes through the washer and the upper plate portion of the upper arm member and is threadably attached to lower plate portion of the lower arm member, such that the upper arm member and lower arm member are pivotably adjustable with respect to one another pivoting around the locking bolt and washer.

3. The system of claim 2, wherein the upper arm member further comprises an elongated slot formed in the upper plate portion passing from a bottom surface to a top surface, such that the washer resides in the elongated slot with the at least one convex exterior surface of the washer facing a wall of the elongated slot, such that the upper arm member and lower arm member are laterally adjustable with respect to one another by placement of the washer and locking at a desired position in the elongated slot.

4. The system of claim 3, wherein the plate portion of the upper arm member includes an inset recess be formed in the lower surface thereof surrounding the elongated slot.

5. The system of claim 4, wherein the inset recess has a substantially vertical sidewall topped by a slanted portion.

6. The system of claim 5, wherein the washer comprises an outer sidewall with opposite parallel portions sized to fit within the vertical sidewall of the inset recess an upper surface that is angled to match the slanted portion of the inset recess.

7. The system of claim 2, wherein the lower arm member further comprises a threaded hole formed in the lower plate portion for attachment to the locking bolt.

8. The system of claim 1, wherein the first swivel rod connector comprises two side arms with a recess therebetween disposed at the distal end of the upper arm and a swivel member disposed within the recess on a pin extending between the two side arms.

9. The system of claim 8, wherein the swivel member comprises an outer surface which curves to a tip and an inner surface which follows a complementary curve from the tip to form a concavity.

10. The system of claim 1, wherein the first locking rod connector comprises an elongated body having a locking portion extending straight from a first end to a rod connection portion which extends at an angle distally from the locking portion to a curved tip.

11. The system of claim 10, wherein the rod connection portion extends at an angle of about 140 degrees from the locking portion.

12. The system of claim 10, wherein the locking portion comprises a threaded hollow shaft opening at an upper surface thereof

13. The system of claim 10, wherein the upper arm further comprises a locking connector port passing generally from an upper surface to a lower surface of the upper arm which is sized to receive the locking portion of the first locking rod connector.

14. The system of claim 1, wherein the second swivel rod connector comprises two side arms with a recess therebetween disposed at the distal end of the lower arm and a swivel member disposed within the recess on a pin extending between the two side arms.

15. The system of claim 14, wherein the swivel member comprises an outer surface which curves to a tip and an inner surface which follows a complementary curve from the tip to form a concavity.

16. The system of claim 1, wherein the second locking rod connector comprises an elongated body having a locking portion extending straight from a first end to a rod connection portion which extends at an angle distally from the locking portion to a curved tip.

17. The system of claim 16, wherein the locking portion comprises a threaded hollow shaft opening at an upper surface thereof

18. The system of claim 16, wherein the lower arm further comprises a locking connector port passing generally from an upper surface to a lower surface of the lower arm which is sized to receive the locking portion of the second locking rod connector.

19. A transverse connector system for coupling elongate elements to each other in a spinal fixation system, comprising:
an upper arm member with an upper plate portion with an elongated slot formed therein and a first connection end;
a lower arm member with a lower plate portion and a second connection end;
a washer with at least one convex exterior surface disposed between the upper arm member and the lower arm member, the washer residing in the elongated slot with the at least one convex exterior surface of the washer facing a wall of the elongated slot, such that the upper arm member and lower arm member are laterally adjustable with respect to one another by placement of the washer at a desired position in the elongated slot; and
a locking bolt, wherein the locking bolt passes through the washer and the upper plate portion of the upper arm member and is threadably attached to lower plate portion of the lower arm member, such that the upper arm member and lower arm member are pivotably adjustable with respect to one another pivoting around the locking bolt and washer.

20. The system of claim 19, wherein the plate portion of the upper arm member includes an inset recess be formed in the lower surface thereof surrounding the elongated slot.

21. The system of claim 20, wherein the inset recess has a substantially vertical sidewall topped by a slanted portion.

22. The system of claim 21, wherein the washer comprises an outer sidewall with opposite parallel portions sized to fit within the vertical sidewall of the inset recess an upper surface that is angled to match the slanted portion of the inset recess.

23. The system of claim 19, wherein the first swivel rod connector comprises two side arms with a recess therebetween disposed at the distal end of the upper arm and a swivel member disposed within the recess on a pin extending between the two side arms and the first locking rod connector comprises an elongated body having a locking portion extending straight from a first end to a rod connection portion which extends at an angle distally from the locking portion to a curved tip.
